(19) United States
(12) Patent Application Publication
(10) Pub. No.: US 2020/0209144 A1
(43) Pub. Date: Jul. 2, 2020

(54) PLASMONIC SENSORS AND METHODS FOR THE DETECTION OF CORNEAL INJURY

(71) Applicant: THE BOARD OF TRUSTEES OF THE UNIVERSITY OF ILLINOIS, Urbana, IL (US)

(72) Inventors: Dipanjan PAN, Champaign, IL (US); Santosh Kumar MISRA, Urbana, IL (US); Leanne T. LABRIOLA, Champaign, IL (US)

(73) Assignee: THE BOARD OF TRUSTEES OF THE UNIVERSITY OF ILLINOIS, Urbana, IL (US)

(21) Appl. No.: 16/640,915

(22) PCT Filed: Aug. 23, 2018
(86) PCT No.: PCT/US2018/047761

§ 371 Date: Feb. 21, 2020

Related U.S. Application Data
(60) Provisional application No. 62/548,997, filed on Aug. 23, 2017.

(51) Int. Cl. G01N 21/01; A61B 3/10; G01N 21/29; G01N 21/552; G01N 33/52; G01N 33/82; G01N 21/65

(52) U.S. Cl. CPC G01N 21/01; A61B 3/101; G01N 21/29; G01N 21/554; G01N 2021/7756; G01N 33/82; G01N 21/658; G01N 2800/16; G01N 33/52

(57) ABSTRACT

The present disclosure provides a method for detection of an analyte in a sample, comprising contacting a biological sample with a biosensor comprising a container or substrate and a gold (III) chloride gel comprising a plurality of gold chloride nanoparticles, determining an optical condition for the sample, and detecting a concentration of the analyte in the sample based on the optical condition. The method can be used for diagnosing an eye condition in a subject. The present disclosure also provides biosensors and kits for the detection of an analyte.

### PRIORITY

This application claims the benefit of U.S. Ser. No. 62/548,997, filed on Aug. 23, 2017, which is incorporated by reference herein in its entirety.

### BACKGROUND

Eye injuries and ocular complications present to many health care professionals through emergency department visits, convenient care appointments or primary care evaluations; however, accurate ocular examination typically requires specialty training and expert knowledge of the use of ophthalmic diagnostic equipment such as the slit lamp biomicroscope. The limited instruction available on these devices and restricted access to the equipment due to the high cost and immobility, inhibit the ability for primary care providers to adequately diagnose, triage, or manage complicated ocular conditions. This is particularly problematic when cases of serious ocular injuries, that require urgent attention, present outside of an ophthalmology office. This occurs in patients with a suspected ruptured globe or post-operative infections.

Current methods for evaluating the integrity of the anterior globe in trauma patients and the wound integrity in post-operative patients involve the use of the Seidel Test. This test is performed by placing a high concentration of fluorescein dye into the ocular tear film and then observing for a change in the color of the dye. The change in color indicates the passage of aqueous humor through a corneal or anterior sderal wound, which represents a direct communication of the internal eye fluid with the external tear film. The Seidel Test is subjective and not standardized, and the amount of pressure and technique used when performing this test varies between clinicians. Other devices that are used to aid in diagnosis of trauma patients include conventional X-ray, computed tomography (CT), ultrasound (US), and magnetic resonance imaging (MRI), but they are limited in their capability to detect eye injuries. Specifically, plain film radiographs have no utility in detecting soft tissue injuries to the eye; CT images do not visualize small anterior lacerations to the cornea, and US is contraindicated with anterior globe ruptures. In addition, all of these imaging devices are expensive and are restricted to hospital settings due to their size and cost. Furthermore, none of these devices are available for evaluation of an eye trauma by first responders in the field or for military use in combat settings.

Compositions and methods are needed in the art for inexpensive, point of care diagnosis of eye injury and disease.

### SUMMARY

One embodiment of the disclosure provides biosensor for the detection of an analyte. The biosensor comprises a container or substrate and gold (III) chloride gel. The analyte can be ascorbic acid. The gold (III) chloride gel can be formed from a gold chloride solution. The gold chloride solution can have a concentration of between about 10 mM and about 50 mM. The container or substrate can be a tube, well, bottle, cylinder, dish, cup, bag, or channeled, textured, or flat plastic or glass sheet.

Another embodiment of the disclosure provides a kit for detection of an analyte in a sample. The kit comprises a biosensor and a collection container for collecting the sample. The kit can also include a color pixel code legend, wherein the color pixel code legend includes colors and respective analyte concentrations. The kit can also comprise a color pixel detection device. The color detection device can comprise a camera and a color picker tool.

Yet another embodiment of the disclosure provides a method of detecting an analyte in a sample. The method comprises contacting a biosensor with the sample, determining an optical condition for the sample, and detecting a concentration of the analyte in the sample based on the optical condition. The analyte can be ascorbic acid. The optical condition can be color intensity or color change. The sample can be tears, tear film, aqueous humor, sweat, blood, serum, plasma, urine, saliva, or other bodily fluids. Determining the optical condition of the sample can occur within between about one to about ten minutes of contacting the biosensor with the sample. Determining the optical condition of the sample can also comprise taking a picture of the gold chloride gel after being contacted with the sample. Determining the optical condition of the sample can also comprise viewing the gold chloride gel after being contacted with the sample. The concentration of the analyte can be reported on an electronic screen. The concentration of the analyte in the sample can indicate an eye condition. The eye condition can be a full or partial thickness laceration or perforation to the anterior chamber of the eye, eye disease, mechanical or chemical eye injury, anterior scleral injury, corneal wound integrity, aqueous humor leaks, eye ulcer, infection, wound healing, or surgical incisions. The concentration of the analyte in the sample can indicate severity of the eye condition or speed of wound healing.

Yet another embodiment of the disclosure provides a method of diagnosing an eye condition in a subject. The method comprises contacting the biosensor of with a sample from the subject, detecting a concentration of an analyte in the sample, and diagnosing an eye condition in the subject where the concentration of analyte is elevated as compared to a control sample. The sample can be tears, tear film, aqueous humor, sweat, blood, serum, plasma, urine, saliva, or other bodily fluids. The eye condition can be a full or partial thickness laceration or perforation to the anterior chamber of the eye, eye disease, mechanical or chemical eye injury, anterior scleral injury, corneal wound integrity, aqueous humor leaks, eye ulcer, infection, wound healing, or surgical incisions.

### BRIEF DESCRIPTION OF THE DRAWINGS

The features, objects and advantages other than those set forth above will become more readily apparent when consideration is given to the detailed description below. Such detailed description makes reference to the following drawings, wherein

FIG. 1. Components of one embodiment of the present disclosure.

FIG. 2. (A) Hydrodynamic diameter of gold chloride reduced with aqueous humor (left) and ascorbic acid (right) 250 μM. (B) TEM of gold chloride reduced with ascorbic acid (50 nm scale bar) 250 μM. (C) TEM of gold chlorides reduced with aqueous humor (50 nm scale bar). (D) UV-Vis spectra of gold chlorides reduced with aqueous humor (top) and ascorbic acid (bottom) 250 μM. (E) Raman spectra of gold chlorides reduced with aqueous humor (top) and ascorbic acid (bottom) 250 μM.

FIG. 3. (A) A schematic describing the correlation of color codes with AA concentration from gold (III) chloride gel reduced with different tear film samples. Characterization of prepared gold (III) chloride gel (B) before and (C) after reduction with AA concentrations. TEM images from reduced gold (III) chloride gel obtained from (D) AA (1 mM; Spot 2). Inset represents calculated Feret diameter of the jelly fish structure and (E) AH incubation (AH1; Spot 4). Inset represents TEM image of one unit of reduced gold as a ‘jelly fish’ morphology. (F) Raman scattering pattern of reduced gold (III) chloride gel with high signature intensity by AH.

FIG. 4. Hexadecimal R values from photographed gold (III) chloride gels as a function of AA concentration (50-2000 μM) in DI-H₂O (A) (n=50 images) and (50-2000 μM) CTF (C) (n=50 images). Hexadecimal R values from photographed gold (III) chloride gels as a function of AA concentration in DI-H₂O (B) (n=50 images) and CTF (D) (n=50 images).

FIG. 5. (A) Concentration of AA in standard samples and correlation with absorption intensity at 490 nm. (B) Concentration of AA in clinical AH samples as calculated from Hexadecimal R values from photographed gold (III) chloride gels as a function of AA concentration in water (n=25 images). AA concentrations of 4.079, 2.247, 5.119, 2.792, and 14.623 nM are obtained in the AH samples #1, 2, 3, 4 and 5 respectively (C) R Hexadecimal values from photographed gold (III) chloride gels as a function of AA concentration in water (n=50 images).

FIG. 6. (A) gold (III) chloride gel tube sample (i) without and (ii) with incubated AA (B) Color code ring with printed AA concentrations, (C) glass tubes for transferring AA samples on gold (III) chloride gel tubes and (DI) AA concentrations calculated (numbers on color ring) from different AH samples calculated using Pixel Picker® app.

FIG. 7. (A) Specificity of gold (III) chloride gel. Actual hexadecimal value of gold (III) chloride gel and after treatment with L-lactic acid, sialic acid, and ascorbic acid. This testing confirms that ascorbic acid can be detected in tear film with high selectivity. Statistical analysis was performed using one-way ANOVA comparing gold (III) chloride gel background results with rest of the samples. (B) Sensitivity of AA detection in DI-H₂O and CTF samples.

FIG. 8. Gold nanoparticles produced after reducing gold chloride sol using aqueous humor (AH) and further changes with impregnated additional ascorbic acid (AA). (A) Hydrodynamic diameter of gold nanoparticles prepared with AA as reducing agent and (B) AH as reducing eye fluid and effect of further AA addition. (C) Surface plasmon resonance generated after reducing gold chloride using AA and (D) AH and effect of further AA addition.

FIG. 9. Visual inspection of gold nanoparticles produced after reducing gold chloride sol in agarose gel using AA, AH and further impregnated additional AA. Eight different samples (20 μL) were used for study.

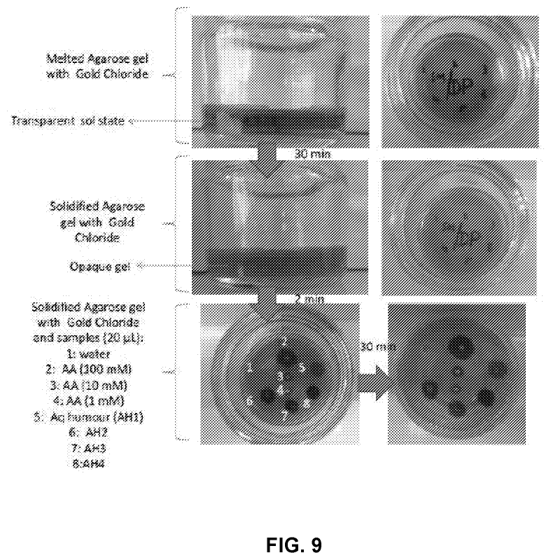

While the present disclosure is susceptible to various modifications and alternative forms, exemplary embodiments thereof are shown by way of example in the drawings and are herein described in detail. It should be understood, however, that the description of exemplary embodiments is not intended to limit the disclosure to the particular forms disclosed, but on the contrary, the intention is to cover all modifications, equivalents and alternatives falling within the spirit and scope of the disclosure as defined by the embodiments above and the claims below. Reference should therefore be made to the embodiments above and claims below for interpreting the scope of the disclosure.

### DETAILED DESCRIPTION

The disclosure provides devices and methods that provide an objective, reliable platform for testing of analytes such as ascorbic acid (AA) within the ocular tear film or other bodily fluids as a surrogate biomarker of for example, anterior sderal or corneal wound integrity, which could replace the subjective Seidel Test and provide more information than the Seidel Test since the device will provide an objective measurement that the Seidel test cannot provide. The method utilizes the difference in AA concentrations found in ocular fluids such as aqueous humor and ocular tear film. Aqueous humor has an average AA concentration of about 1049±433 micromol/L whereas the ocular tear film only has an average AA concentration of about 23±9.6 micromol/L. With this fundamental difference in concentration and the fact that aqueous humor is continuously produced within the anterior chamber, when the integrity of the anterior globe is disturbed from a laceration, the higher concentrations of AA from within the continuously flowing aqueous humor will be released into the tear film causing a rise in the amount of AA in the tear film that can be quantified. The tear film AA concentration can be detected and measured by devices and methods of the disclosure.

There is currently no FDA-approved point-of-service (POS) tests that directly measure analytes such as ascorbic acid (AA) in tear film. Other methods of AA detection include HPLC, electrochemical, colorimetric, absorbance, and fluorescence measurement, but all of them have serious limitations of requirement of sophisticated instrumentation, limitation to low concentration detection, and extensive sample preparation.

TABLE 1
Current AA detection methods.

| Method/Transducer | Sample Type |
|---|---|
| Resistance change, Graphene platelet | Aqueous humor |
| HPLC-UV | Blood Plasma |
| HPLC-UV | Seminal Plasma |
| Electrochemical, screen-printed | AA standards |
| electrode | |
| Dissolved oxygen probe | Fruit juice |
| ISFET, MnO₂ nanoparticles | AA standard |
| ISFET, peroxidase | AA standard |
| Impedance, graphite | Fruit juice |
| Potentiometric, ZnO nanorod | AA standard |
| Electrochemical (cyclic voltammetry), | AA standard |
| Nitrogen doped graphene | |

The measurement provided by certain biosensor devices of the disclosure can be performed in the clinical setting with an immediate result without having to send the samples to a laboratory for further sampling or analysis, as competing assays require. In addition, a kit including the biosensor can report the level of AA concentration on an electronic screen. This feature of a biosensor enables clinical use since analytes such as AA can rapidly degrade after collection. In previous studies L-ascorbic acid solution degraded during storage for longer periods in the presence of oxygen due to oxidation of AA. It involves the loss of two electrons and two protons while oxidation product dehydroascorbic acid (C₆H₆O₆) is relatively unstable in aqueous solution since it spontaneously reacts with water to yield 2,3-diketogulonic acid. The rate of oxidation depends on the concentration of oxygen, temperature, enzyme or transition metal catalysis or basic pH abundance. The ability of the biosensor of the disclosure to test the tear film immediately avoids the problems that occur with oxidation and increases accuracy of the test. The disclosure presents examples of point-of-care biosensor devices, which can accurately and quantitatively measure analytes such as ascorbic acid levels in samples such as human tear film. The measurement of analytes such as ascorbic acid can be an important biomarker for the stability of the cornea integrity. A further benefit is an easy to use device that can utilize disposable biosensor strips. Devices and methods of the disclosure can supplement clinical diagnosis and provide valuable information for first responders and quantitative, objective measurements for degree of injury.

Biosensor

Devices of the disclosure include biosensors for the detection of analytes such as ascorbic acid. The biosensor can comprise a container or substrate and gold (III) chloride gel. The gold (III) chloride gel may be formed from a gold chloride solution. In an example embodiment, the gold chloride solution may have a concentration of between about 10 mM and about 50 mM (for example, about 10, 20, 30, 40, 50, or more mM). In other embodiments, the gold chloride solution may have a concentration of between about 20 mM and about 40 mM. In further embodiments, the gold chloride solution may have a concentration of about 30 mM.

The gel can be a hydrogel, organogel, xerogel, or nanocomposite gel. A hydrogel is a two- or multi-component system comprising a three-dimensional network of polymer chains and water that fills the space between macromolecules. Hydrogels can be made by several methods including, for example, one-step procedures like polymerization and parallel cross-linking of multifunctional monomers. Multiple step procedures, for example, synthesis of polymer molecules having reactive groups and their subsequent cross-linking or reacting polymers with suitable cross-linking agents can also be used. A hydrogel can be a natural or synthetic hydrogel. A hydrogel can be a homopolymeric, copolymeric, diblockcopolymeric, triblockcopolymeric, multipolymer interpenetrating polymeric, amorphous (non-crystalline), semicrystalline, crystalline hydrogels. In an example a gel can comprise collagen, gelatin, agar, agarose, acrylamide, acrylic acid, dextran, hyaluronic acid, pectin, alginate, or poly(acrylic acid).

In an example embodiment, the biosensor can comprise a container for holding the gold chloride (III) gel. The container may be a fluidic chamber, tube, well, bottle, cylinder, dish, cup, or bag. The container can accommodate a fluid volume of about 0.1, 0.5, 1.0, 5, 10, 15, 20, 50, 100 μL or more (or any range between about 0.1 and 100 μL). The container can be made of plastic, glass, or any other suitable material.

In another example embodiment, the gold (III) chloride gel can be present on a substrate, such as a channeled, textured, or flat sheet. The substrate can comprise one or more of an acrylamide, cellulose, nitrocellulose, glass, indium tin oxide, silicon wafer, mica, polystyrene, or polyvinylidene fluoride (PVDF) filter, filter paper (e.g., Whatman), glass fiber filters (GF), fiberglass, polyethylimine coated GFs, porous mylar or other transparent porous films, cellulose nitrate (CN) membrane, mixed cellulose ester membrane, cellulose acetate membrane, polyethersulfone (PES) membrane, PTFE membrane, ultrafiltration membranes of poly(vinyl chloride) (PVC), carboxylated poly(vinyl chloride) (CPVC), polystyrene, polyethylene vinyl acetate, polypropylene, polymethacrylate, polyethylene, polyethylene oxide, polysilicates, polycarbonates, teflon, fluorocarbons, nylon, silicon rubber, polyanhydrides, polyglycolic acid, polylactic acid, polyorthoesters, polypropylfumerate, collagen, glycosaminoglycans, and polyamino acids. The substrate can be formed into pre-perforated strips, individual strips, individual sheets, or any other suitable shape.

The biosensor can comprise a cover comprised of plastic or other suitable material. The cover can protect the gold (III) chloride gel within the container or on the substrate. A cover can be present on top of a container or substrate.

A biosensor of the disclosure can disposable.

Analytes

Analytes can be, for example, ascorbic acid, sialic acid, beta-caroten, lutein, lycopene, selenium, vitamin A, vitamin C, vitamin E or other antioxidants. That is, any of these analytes can be detected as described herein for ascorbic acid.

In one embodiment an analyte is present in a biological sample such as be tears, tear film, aqueous layer of the tear film, aqueous humor, sweat, blood, serum, plasma, urine, saliva, or other bodily fluids.

An analyte can be present in tear film or aqueous humor. In one embodiment, an analyte is present in both the aqueous humor and in the tear film, but in a normal, non-diseased or non-injured subject is present in the aqueous humor at a higher concentration than in the tear film. In another embodiment, the analyte is present in the aqueous humor and is not present in the tear film in a normal, non-diseased, non-injured subject. When an eye condition is present, the amount of analyte increases in the tear film due to leakage or movement from the aqueous humor to the tear film.

Kits

The disclosure also provides a kit comprising a biosensor and a collection container for collecting a sample. The biosensor can comprise a container or substrate and gold (III) chloride gel. The collection container can be any container capable of collecting and holding the sample, such as collection strips, syringes, or collection tubes.

The kit can also comprise a color code legend, such as a color pixel code legend, or a color ring including a range of colors and their respective analyte concentrations. Based on optimized information, a color code legend was developed with assigned shade of color representing a nominal gold reduction and related AA concentration

A kit can further comprise a color detection device, such as a color pixel detection device. The color detection device can comprise a color strip, such as a piece of paper, that can be held up to the container or substrate in order to visually examine colors. In other embodiments, the color detection device can comprise an instrument for measuring color, such as a colorimeter, a spectroradiometer, a spectrophotometer, a spectrocolorimeter, a densitometer, a color temperature meter, or other commonly used devices. A colorimeter measures the absorbance of wavelengths of light. A spectroradiometer measures the absolute spectral radiance or irradiance of a light source. A spectrophotometer measures the spectral reflectance, transmittance, or relative irradiance of a color sample. A spectrocolorimeter is a spectrophotometer that can calculate tristimulus values. A densitometer measures the degree of light passing through or reflected by a subject. A color temperature meter measures the color temperature of an incident illuminant. A device for Raman spectroscopy can also be used, such as a Raman microscope, which is a standard optical microscope with an excitation laser, laser rejection filters, a spectrometer or monochromator, and an optical sensitive detector such as a charge-coupled device or photomultiplier tube.

In an example embodiment, the color detection device can comprise a camera and a color picker tool, such as Pixel Picker®. The color detection device may be part of a computer, a hand-held device, a cell phone, and a tablet. The color detection device can provide information (e.g., a sample identifier, a subject identifier, a quantity detected of one or more analytes, a positive or negative reading regarding the presence or absence of an analyte, or a combination thereof) to a data acquisition system, which can then analyze the information and provide an easy to read and interpret result. The data acquisition system can also be included in the kit.

A kit can further comprise a screen that allows for visualization of an amount of an analyte, such as ascorbic acid, present in a sample.

A kit can be battery operated.

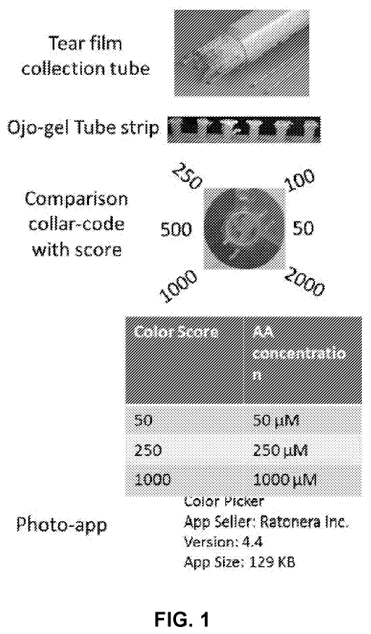

An example kit comprising a biosensor, a collection container, a color legend, and a color detection device is illustrated in FIG. 1.

In other embodiments, the kit can comprise a plurality of biosensors and a plurality of collection containers.

Methods of Detection of Analytes and Diagnosis

The disclosure provides methods for detecting an analyte in a sample comprising contacting a biosensor with the sample, determining an optical condition for the sample, and detecting a concentration of the analyte in the sample based on the optical condition. The analyte can be ascorbic acid. The optical condition can be color intensity or a change in color. The color intensity or color change can be proportional to the concentration of the analyte. The sample can be tears, tear film, aqueous layer of the tear film, aqueous humor, sweat, blood, serum, plasma, urine, saliva, or other bodily fluids.

Contacting the biosensor can comprise touching the surface of the gold (III) chloride gel with the sample.

In an example embodiment, the analyte, e.g., ascorbic acid, reduces the gold (III) chloride gel to gold nanoparticles and the gold nanoparticles produce the optical condition. Nanoparticles are particles between 1 and 100 nanometres (nm). Gold nanoparticles absorb and scatter light resulting in colors ranging from vibrant reds to blues to black and finally to clear and colorless, depending on particle size, shape, local refractive index, and aggregation state. The change in color or color intensity can be directly correlated to the amount or concentration of analyte in the sample. That is, a particular color or color intensity can represent a certain amount or concentration of analyte in the sample. One of skill in the art can determine a color condition standard for each type of analyte and/or sample.

TEM (a, b, and c) images of prepared mesoporous silica nanoparticles with mean outer diameter: (a) 20 nm, (b) 45 nm, and (c) 80 nm. SEM (d) image corresponding to (b). The insets are a high magnification of mesoporous silica particle.

Determining the optical condition of the sample can occur in less than about ten minutes. In an example embodiment, determining the optical condition of the sample can occur in between about one and about two minutes.

Determining the optical condition can comprise taking a picture of the gold (III) chloride gel after being contacted with the sample. Determining the optical condition can also comprise viewing the gold (III) chloride gel after being contacted with the sample. The optical condition can be directly correlated to the amount or concentration of analyte in the sample. The amount or concentration of the analyte can be reported on an electronic screen.

The concentration of the analyte in the sample can indicate an eye condition. The eye condition can be a full or partial thickness laceration or perforation to the anterior chamber of the eye, eye disease, mechanical or chemical eye injury, anterior scleral injury, corneal wound integrity, aqueous humor leaks, eye ulcer, infection, wound healing, or surgical incisions.

The presence, absence, or an amount or concentration of an analyte in the sample can be detected. The amount of an analyte, such as ascorbic acid, can indicate the severity of the eye condition. That is, the higher the amount of analyte, e.g., ascorbic acid, the greater the severity of the disease or condition (e.g., an eye condition). Unlike previous methods, this method allows for a measurement of AA that is quantitative and not merely qualitative.

A higher amount or concentration of the analyte in the sample can correlate with better wound healing, for example, in wounds such as surface abrasions (e.g., corneal ulcers) or post-surgical incisions.

The disclosure also provides methods for detecting an eye condition in a subject comprising detecting the level or amount of ascorbic acid in a tear film sample from a subject. The level or amount of ascorbic acid in a tear film can be compared to a control sample or control standard. If an elevated level of ascorbic acid is present in the tear film, then a medical practitioner can further evaluate to determine whether medical or surgical treatment is needed. For example, an abnormal amount of AA in the tear film following an eye injury may alert the medical practitioner that further evaluation is needed, and if surgical repair is needed. In the case of post-surgical wounds, the amount of AA in the tear film can be higher than normal shortly after surgery, but typically returns to a level closer to a control standard within a week after surgery. If higher levels of AA persist past one week or suddenly increase, the medical practitioner is alerted that further evaluation and possible medical or surgical treatment is needed.

The disclosure provides methods of diagnosing an eye condition. The eye condition can be a state of the eye, a disease, or an injury. The method comprises contacting a biosensor with a tear, tear film, or aqueous humor sample of a subject and detecting an amount of an analyte in the sample. An eye condition is diagnosed in the subject where the concentration of the analyte in the sample is elevated as compared to a control sample or control standard. The eye condition can be selected from a full or partial thickness laceration or perforation to the anterior chamber of the eye, eye disease, mechanical or chemical eye injury, anterior scleral injury, corneal wound integrity, aqueous humor leaks, eye ulcer, infection, wound healing, or surgical incisions.

In one embodiment of the disclosure, a biosensor of the disclosure can be used to detect an analyte such as ascorbic acid in other types of samples such as sweat, blood, serum, plasma, urine, saliva, or other bodily fluids to diagnose or detect other conditions.

Analytes such as ascorbic acid can be found in higher amounts or levels in injured or diseased samples (e.g., a sample of a subject with an eye condition) as compared to control subject samples from non-injured or non-diseased subjects. The relative levels of analytes, such as ascorbic acid, in subject samples can indicate progression of disease and disease severity. That is, in some instances, a greater amount or level of analyte in a test sample means a more severe disease state or condition.

Elevated levels of analytes, such as ascorbic acid, are levels that are about 10, 20, 30, 40, 50, 60, 70, 80, 90, 100, 200, 300, 400, 500% or more than control samples or control standards. Elevated levels of analytes such as ascorbic acid are levels that are about 10 to 500% more; about 20 to 500% more; about 30 to 500% more; about 40 to 500% more; about 50 to 500% more; about 60 to 500% more; or about 100 to 500% more than control samples or control standards.

Elevated levels of analytes such as ascorbic acid can also be levels that are statistically significantly increased amounts when compared to control samples or control standards.

Elevated levels of ascorbic acid can also be about 40, 50, 60, 70, 80, 90, 100, 200, 300, 400, 500, 600, 700, 800, 900, 1,000, 1,100, 1,200, 1,300, 1,400 or more micromol/L (or any range between about 40 and about 1,400 micromol/L, for example about 50 to about 1,400; between about 100 and about 1,400, between about 500 and about 1,400 micromol/L). Control levels or control standards of ascorbic acid can be about 50, 40, 30, 20, 10 or less micromol/L (or any range between about 10 and about 50, between about 10 and about 40, between about 10 and 30, or between about 10 and 20 micromol/L).

Elevated levels of analytes such as ascorbic acid can be compared to control samples or control standards that are determined using normal control subjects who do not have any type of disease, eye disease, or eye condition. For example, the level can be compared to the level in the contralateral eye.

In some embodiments, the level of analytes such as ascorbic acid in a test sample is compared the level of the analyte in a control sample from one or more normal control subjects. Typically, the measured control level in the control sample is then compared with the analyte level measured in the test sample. Alternatively, the level of an analyte such as ascorbic acid in the test sample is compared to a previously determined or predefined control level (a “control standard”). For example, the control standard for an analyte such as ascorbic acid of can be calculated from data, such as data including the levels of the analyte in control samples from a plurality of normal control subjects. The normal control subjects and the test subject under assessment can be of the same species.

In an example embodiment, the method can include: 1) download color picker app on a phone, 2) take out one gold (III) chloride gel tube from a strip, 3) take one tear film collection tube and collect tear film from eye, 4) release tear film on gel by touching the surface, 5) wait for one minute, 6) take picture of gel from top of the tube using color picker app, 7) compare colors of gel and collar-code, and 8) acquire the AA level.

As used herein, “patient” or “subject” means an individual having symptoms of, or at risk for, eye disease, eye condition, or eye injury, or other disease, condition, or malignancy. A patient may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human such as canine, feline, bovine, equine, or rodent) that may benefit from the methods and compositions contemplated herein.

Results (i.e., the presence, absence, or amount of an analyte) can be delivered with about 10, 5, 4, 3, 2, 1 minutes or less using only small sample sizes (e.g., about 50, 20, 15, 10, 5, 2, 1.0, 0.5 or less μL).

Unless otherwise explained, all technical and scientific terms used herein have the same meaning as commonly understood by one of ordinary skill in the art to which a disclosed disclosure belongs. The singular terms “a,” “an,” and “the” include plural referents unless context clearly indicates otherwise. Similarly, the word “or” is intended to include “and” unless the context clearly indicates otherwise. “Comprising” means “including”; hence, “comprising A or B” means “including A” or “including B” or “including A and B.”

While the present disclosure can take many different forms, for the purpose of promoting an understanding of the principles of the disclosure, reference will now be made to the embodiments illustrated in the drawings and specific language will be used to describe the same. It will nevertheless be understood that no limitation of the scope of the disclosure is thereby intended. Any alterations and further modifications of the described embodiments, and any further applications of the principles of the disclosure as described herein are contemplated as would normally occur to one skilled in the art to which the disclosure relates.

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the disclosure. Thus, it should be understood that although the present disclosure has been specifically disclosed by preferred embodiments, exemplary embodiments and optional features, modification and variation of the concepts herein disclosed may be resorted to by those skilled in the art, and that such modifications and variations are considered to be within the scope of this disclosure as defined by the appended aspects. The specific embodiments provided herein are examples of useful embodiments of the present disclosure and it will be apparent to one skilled in the art that the present disclosure may be carried out using a large number of variations of the devices, device components, methods steps set forth in the present description. As will be obvious to one of skill in the art, methods and devices useful for the present methods can include a large number of optional composition and processing elements and steps.

The embodiments illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms, while retaining their ordinary meanings.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the aspects herein. Ranges can be expressed herein as from “about” one particular value, and/or to “about” another particular value. As used herein, the term “about” in association with a numerical value means that the numerical value can vary plus or minus by 5% or less of the numerical value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

### EXAMPLES

### Example 1

Synthesis of Gold (III) Chloride-Agarose Gel

A gold (III) chloride-agarose gel was developed by dissolving 23.1 mg of gold (III) chloride hydrate 99.99% salt in 2.3 ml of DI-H₂O to obtain a final concentration of 30 mM. Then 1 ml of 30 mM gold (III) chloride solution was mixed with 160 mg of agarose powder in 4 ml of DI-H₂O in a 20 ml disposable scintillation vial. The total solution of 5 ml was then heated in a microwave for 90 seconds. The hot solution was immediately transferred into centrifuged tubes and a capillary tube was placed to create a small channel in the middle of the gel. The solution was allowed to solidify at room temperature (RT) for around 30 minutes and was later stored at 4° C. until further use.

Ascorbic Acid Detection Using Commercial Enzyme-Linked Immunosorbent Assay (ELISA) Kit

ELISA experiments were performed using a 96-well plate. Six known concentrations (0, 2, 4, 6, 8, 10 nmol) of AA and five human AH samples (1-5) with unknown AA concentration were pipetted into the microliter plate wells with a volume of 120 μl (AA/AH Sample Plus AA Assay Buffer) of each sample. 30 μl of catalyst was then added to each standard and sample well. After 15 minutes incubation at RT (18-25° C.), 50 μl of the reaction mix was added to each well containing the AA standard and test samples. A color (pink) was developed within 3 min and was stable for an hour. After 30 minutes, each well was then measured for 2 seconds in a spectrophotometer with Gen 5.0 software at 490 nm OD. Measurements were tested in duplicate sets, and the average value was then utilized to determine the final AA concentration.

Quantification of Gold (III) Chloride Reduction and Correlation to Ascorbic Acid Concentration

To determine out the reducing activity of AA on gold (III) chloride solution to form gold nanoparticles a series of trials were conducted in which pictures were taken with a normal cellphone. Initially, a standard AA solution with concentrations of 2,000 μM, 1,000 μM, 500 μM, 250 μM, 125 μM, and 50 μM was made. The standards were then delivered to the channel created in the gel with help of another capillary glass tube to get some color change which is indicative of reducing activity of AA on gold (III) chloride solution. The gels showed different color changes when reacted with samples of different concentrations. To establish a scale, the reacted PCR tubes were put together in a PCR stand. Several pictures (50) were taken for each concentration from one side with the varying distance of a flash light. An computer application (Pixel Picker® available on Apple App Store®) was used to determine the RGB (red, green, blue) value in the color changed areas. (An RGB color value is specified with red, green, and/or blue. Each parameter (red, green, and blue) defines the intensity of the color as an integer between 0 and 255. For example, RGB (0, 0, 255) is rendered as blue, because the blue parameter is set to its highest value (255) and the others are set to 0.) The same statistics were analyzed for 50 pictures and the average of the RGB values was found for each color changed area. A color sequence was then restored based on the calculated RGB average and correlated with the standard concentration of AA. Based on this optimized information, a color code ring was developed with assigned shade of color representing a nominal gold reduction and related AA concentration. Gold (III) chloride gel tubes were placed in these color rings before capturing snaps of changed color in the gel tubes. Color changes were co-related with used AA concentration.

Extraction of Nanoparticles from Gold (III) Chloride Gel Via Sodium Hypochlorite Based Dissolving Method

Identified portions of reacted gold (III) chloride gel were first removed via stainless steel spatula and placed into 1.5 mL microcentrifuge tubes. To the microcentrifuge tubes containing reacted gold (III) chloride gel, 1 mL of 15% w/v sodium hypochlorite solution admixed, sealed, and incubated for 45 minutes at 65° C. to dissolve the gel. The dissolved gel (65° C.) was then diluted 1:100 fold in room temperature DIH₂O before Raman spectroscopy or TEM measurements.

### Example 2

Feasibility of Plasmonic Gold Nanoparticle Formation by Free Ascorbic Acid and Ascorbic Acid in Aqueous Humor

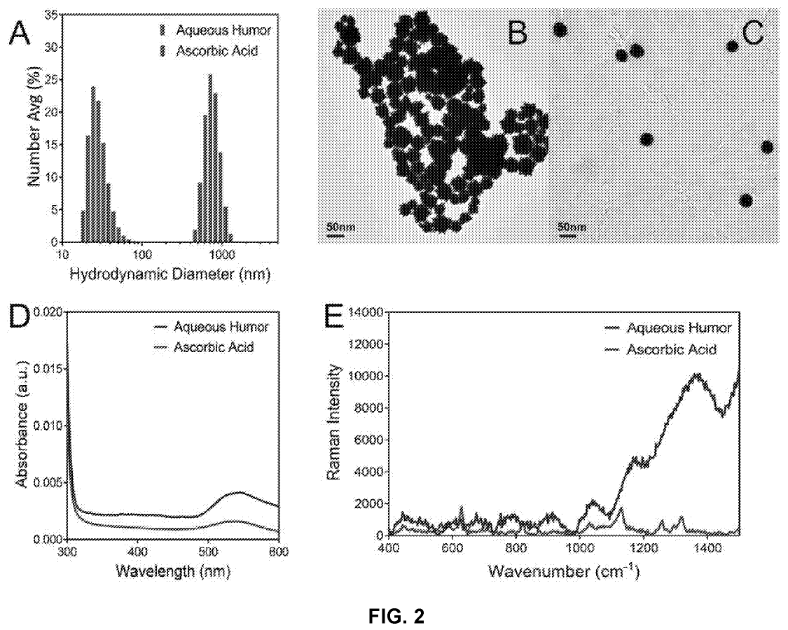

Aqueous humor (AH) contains a wide variety of surfactants as well as containing high concentrations of AA, which can reduce gold (III) as an endogenous reductant to produce gold nanoparticles. An initial screening was used in order to determine the feasibility of designing a plasmonic sensor-based detection of diffuse AA content in controlled solutions (containing only AA and water) with and more complex clinical samples. To determine this feasibility, ionic gold (Au3+) was admixed with either clinically acquired AH or AA at a concentration of 1 mM dissolved in DI-H₂O. From these two mixed solutions, colorimetric reactions occurred—the solution containing AH went from yellow to red with UV-Vis absorbance peak at ˜540 nm (characteristic of gold nanoparticles), and from yellow to clear (with visible brown aggregates) with an absorbance spectrum identical with pure water from the solution with isolated AA (FIG. 2D). Through dynamic light scattering, the mixture with isolated AA resulted in entities of sizes much larger (>500 nm) than those produced with AH (˜40 nm) (FIG. 2A). However, by utilizing transmission electron microscopy (TEM) it was determined that these larger entities were aggregations of nanoparticles ˜40 nm in diameter (FIG. 2B). From this information, it was concluded that AA alone is sufficient to reduce the ionic gold but not to stabilize the nanoparticles enough to keep them segregated. AH, containing both reductant and stabilizing molecules resulted in colloidally stable nanoparticles (˜30 nm anhydrous diameter through TEM) without aggregation (FIG. 2C). These results, indicating that gold nanoparticle reduction occurs via AA in pure solution and as component of AH were further corroborated via surface enhanced Raman spectroscopy. To collect enhanced Raman spectra, mixed solutions of ionic gold with AA in pure solution and as component of AH were pipetted up and down to mix before being pipetted onto glass slides. Solutions on these slides were dried for >24 hours before Raman measurements were collected using a 635 nm laser. For the mixture containing only AA and ionic gold enhancement of AA signature without interference from other components is seen (FIG. 2E). This enhancement found in the samples containing only AA and ionic gold is due to the nano-scaled features of the nanoparticle aggregates acting as “nanoantennae.” Comparing this spectra with the mixture containing AH, much greater signal noise is observed, as well as increase in overall Raman signal. This increase in intensity of Raman signal is causal of there existing a larger population of nanoparticles whose plasmonic absorbance matches the Raman laser wavelength. The Raman signal noise is due to the increased complexity of the mixture resulting from multiple molecules (not just AA) receiving plasmonically enabled Raman signal enhancement. Additionally, AA saturation concentration of gold chloride reduction was achieved by performing a UV-Vis experiment where different titrated amounts of AA (2 μM, 20 μM, 100 μM, 200 μM, 500 μM, 1000 μM and 2000 μM) were used to reduce 3 mM of gold chloride solution. The intensity of plasmonic peak at 540 nm increased with increase in concentration of AA. The peak intensity was found to be saturated at 1000 μM. This indicates that 1000 μM of AA is enough to reduce 3 mM gold (III) chloride solution. This led to use of 30 mM gold chloride solution in preparation of gold (III) chloride gel for providing a wider range of AA in standard and patient samples. These findings support the notion that bodily fluids containing endogenous reductants (such as AA) that can reduce ionic gold, forming plasmonic gold nanoparticles, could potentially be measured in quantifiable manner through optical colorimetric means.

Synthesis of Gold (III) Chloride Gel and Detection of Ascorbic Acid Via Optical Differentiation

It was established that gold chloride could be reduced with ophthalmic fluids known to contain high AA concentration and AA alone, however, AA was not effective in producing colloidally stable gold nanoparticles without stabilizing agents. To address the issue of nanoparticle aggregation in technical grade standards (solutions free of non-analyte biomolecules which are necessary for calibrations) ionic gold was embedded within an agarose gel scaffold as a plasmonic sensing platform where AA (or any other endogenous biological reductant) could be optically quantified (FIG. 2A).

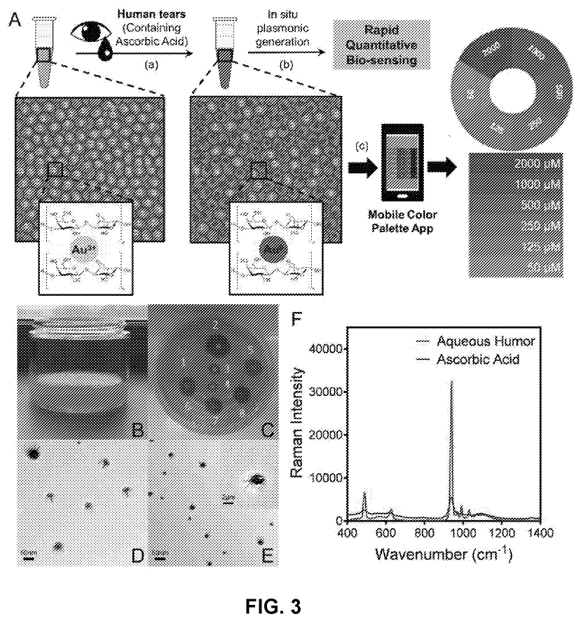

Gold (III) chloride-agarose gel was developed by dissolving 23.1 mg of gold(III) chloride hydrate 99.99% salt in 2.3 ml of DI-H₂O to obtain a final concentration of 30 mM. 1 ml of 30 mM gold (III) chloride solution was admixed with 4 ml of 4% (w/v) agarose in DI-H₂O. The mixtures were microwaved for 90 seconds (high power setting, using a conventional Sunbeam) microwave), until the solution was homogenous with care to ensure that the ionic gold not be microwaved for too long (causing auto-reduction). As the mixture became homogenous, it was poured and cast in 0.6 mL centrifuge tubes for convenience (however the gel can be cast into any number of shapes/forms). Once cooled and solidified, the cast gels were stored at 4° C. prior to experimental measurement (FIG. 3B). To activate the gel as a sensor for biologically endogenous reductants (AA), treatments were applied through channels made with glass capillary tubes. Upon treatment of gel with AA or AH containing AA, the color of the gel would change to a mahogany/orange-brown due to an increased localized nanoparticle formation at the treatment site and could be correlated with color codes (FIG. 3A). Gel sample (FIG. 3B) was incubated (5-30 minutes) with different AA and AH samples generating gold nanoparticles and related plasmonic color (FIG. 3C). Here spots 1-8 represent various volume and concentrations of AA in standard solution as spot 1 for 0 mM (5 μL), spot 2 for 1 mM (5 μL), spot 3 for 10 mM (5 μL) and spot 4 for 100 mM (20 μL) while AA in different AH samples (20 μL) as spot 5 for AH1, Spot 6 for AH2, Spot 7 for AH3 and Spot 8 for AH4. This plasmonic nanoparticle formation resultant of treatment was confirmed through TEM images of nanoparticles extracted from gel spots from AA (1 mM; Spot 2) (FIG. 3D) and AH incubation (AH1; Spot 5) (FIG. 3E) dissolved with sodium hypochlorite solution. To determine the logarithmic colorimetric relationship, 50 pictures were captured of each titrated AA treatment (2000, 1000, 500, 250, 100 and 50 μM) using a camera phone (Apple iPhone 7 (12 MP, f/1.8, 28 mm, ⅓″, phase detection autofocus, OIS, quad-LED dual-tone flash), and the average RGB values were numerically determined using the Pixel Picker® application (available from the Apple App Store®).

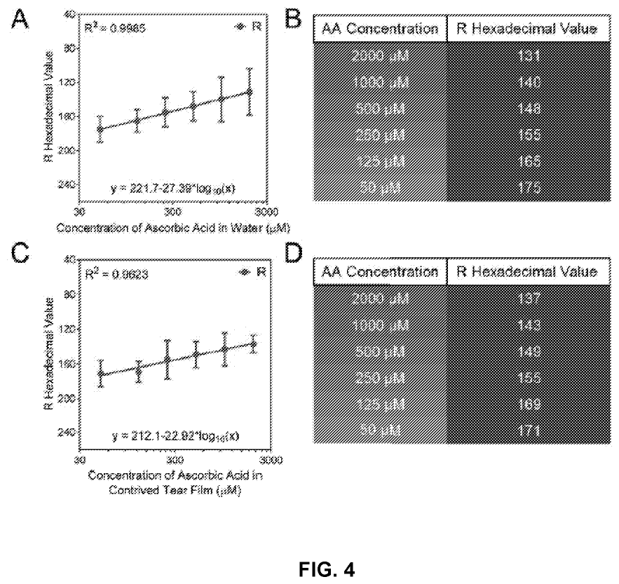

To discover if this colorimetric relationship would be maintained in more complex mixtures, contrived tear film (CTF) was used in place of DI-H₂O for titrated treatments of identical AA concentrations. The contents of CTF, being similar in content to biological tear film, provided an outlet for realistic testing conditions for the gold (III) chloride gel without the volume limitations and variability found in clinical samples. From these treatments with titrated amounts of AA, a logarithmic relationship between the R hexadecimal values of the gold (III) chloride gel and the AA concentration in DI-H₂O (FIGS. 4A, B) CTF samples (FIGS. 4C, D) was found.

Detection of Ascorbic Acid in Aqueous Humor and Validation Against ELISA

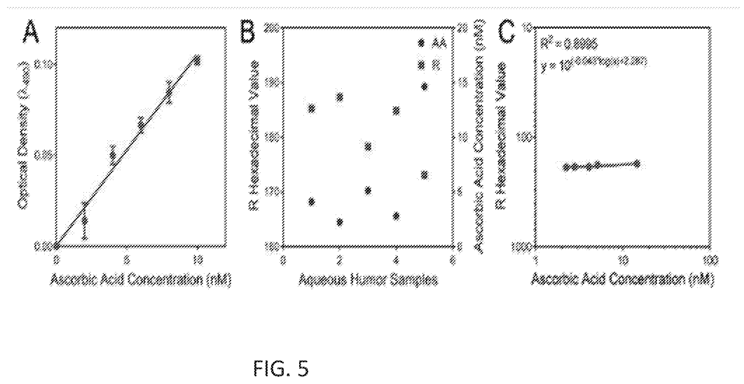

To validate gold (III) chloride gel as a sensor for the detection of AA in clinically relevant settings, AH samples were clinically obtained and then tested for AA using both gold (III) chloride gel and ELISA (FIGS. 5A, B, C). AA concentration of five clinical AH samples were measured in duplicate via ELISA. The calibration of the ELISA against AA was performed in duplicate by dissolving known concentrations of AA in DI-H₂O. The AA levels in AH samples collected from subjects #1, 2, 3, 4 and 5 are 4.079, 2.247, 5.119, 2.792, and 14.623 nM, respectively. AH samples remaining after ELISA measurement were used to treat gold (III) chloride gel and for each of these treatments 50 images with corresponding RGB values were collected. The red (R) hexadecimal value for the gold (III) chloride gel treated with clinical samples had an apparent logarithmic relation to concentration of AA (as determined by ELISA). A comparison of two methods—gold (III) chloride gel and ELISA was done to demonstrate the accuracy (% error) based on the AA level determined in the AH samples. When the red (R) hexadecimal values of the gold (III) chloride gel treated with clinical samples were used to back calculate the AA concentration using the relationships calibrated in DI-H₂O and CTF, a logarithmic relationship within the testing range was found, however, at concentrations higher than those obtained via ELISA. The two methods co-related with regression value (R²=0.8995) and showed strong validity of the proposed gold (III) chloride gel for the detection of AA concentration (FIG. 5C).

Color Code Rings and Simultaneous Calculation of AA Concentration in Aqueous Humor (AH) Samples

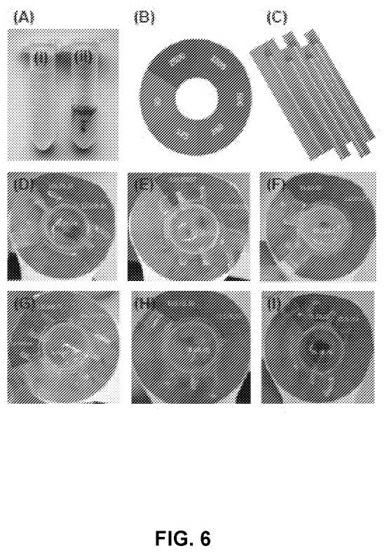

Prepared color rings with known AA concentration color (FIG. 6B) were decorated around the neck of gold (III) chloride gel sample tubes (FIG. 6A) before capillary tubes (FIG. 6C) mediated transfer of AA samples (FIGS. 6E-I) on top of gel tubes. Within two minutes of incubation, a color change was noticed on the top of the gel surface. Pixel Picker® app was used to catch the color and correlated with color codes on rings present around the neck of gel tubes. A range of 50 to 2000 μM of AA was reported in different clinical AH samples.

Specificity and Sensitivity of Gold (III) Chloride Gel

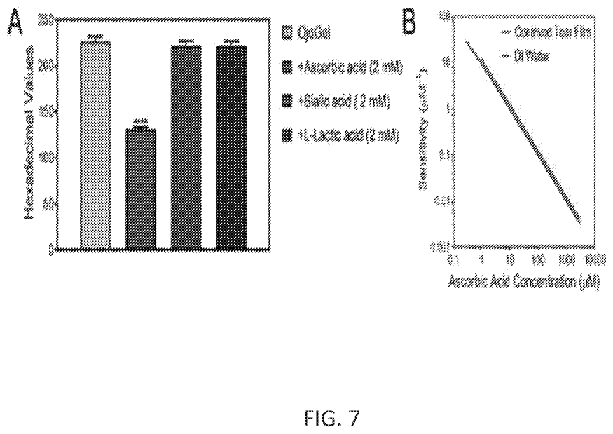

In our experiments, two other major components of AH, L-lactic and sialic acid, were chosen to investigate the specificity of the gold (III) chloride gel. In a typical experiment, 2000 μM AA, L-lactic and sialic acid spiked in contrived tear film (CTF) were added to the gel. It was observed that with the treatment of gel with AA the color of the gel would change to a mahogany/orange-brown due to an increased localized nanoparticle formation at the treatment site but remained the same when treated with CTF spiked with L-lactic acid and sialic acid (FIG. 7A). Composition of the CTF includes Potassium Chloride (KCl), Sodium Chloride (NaCl), Sodium Bicarbonate, Urea, Ammonia Chloride, Y-globulins, Vitamin C, Citric Acid, Albumins (Human), Lysozymes, Pyruvic Acid, Lactic Acid, Hydrochloric Acid, Free Fatty Acids; Wax Esters; Cholesterol Esters; Diesters; Mucin; Free Sterols; Triglycerides; Glycerophospholipids; Sphingophospholipids; Fatty Acids and Hydrocarbons and was found to generate no significant difference in color of gold (III) chloride gel due to these components. To determine this colorimetric relationship again 25 pictures were captured of each titrated acid treatment using a camera phone, and the average RGB values were numerically determined using the Pixel Picker® application (available from the Apple App Store™). The specificity of the gold (III) chloride gel was found to be statistically significant over other acids and the high specificity of the gold (III) chloride gel is mainly due to the high reducing activity of AA. These results manifest that AA can be detected in tear film with high selectivity. To discuss the sensitivity of the gold (III) chloride gel, one must first consider that since resulting colorimetric changes are logarithmically responsive to the concentration of endogenous reductant (AA) in both pure and complex solutions (FIG. 5 and FIG. 6), that the sensitivity (RHex/[AA] (μM⁻¹)) of these measurements will be directly dependent on the treatment concentration (FIG. 7B). This colorimetric relationship, is beneficial in that it is similarly logarithmically responsive like the human eye in perceiving light, in that it provides higher sensitivity between lower treatment concentrations, and that it is more able to detect lower concentrations of reductant in more complex solutions. To calculate the sensitivity (μM⁻¹) for each theoretical treatment concentrations, a first order derivative of the dose-response curves calibrated in DI-H₂O and CTF was taken (FIG. 4). As the relationship between sensitivity (RHex/[AA] (μM⁻¹)) and AA concentration is inverse, one only needs to calculate the detection limit to calculate the maximum sensitivity provided by the gold (III) chloride gel. To calculate the lowest theoretical detectable AA concentration, the value of 255=R is inputted into the logarithmic lines of best fit as calibrated in DI-H₂O and CTF. After calculating the theoretical detection limit, one could also use this to calculate the maximum sensitivity (as described earlier). To determine the effective range of usable concentrations for the biosensor, one needed to determine at what point one would consider the signal to be saturated (when the sensitivity is too low to differentiate between measurements). The saturation sensitivity was arbitrarily set as 0.2 μM⁻¹, as the Pixel Picker® phone application cannot distinguish hexadecimal values with separation less than one. Having chosen this saturation sensitivity, one could then back-calculate the concentration of AA resulting in those sensitivities for both AA in tear film and water, and then use this to calculate the saturation R value.

Furthermore, the repeatability of the gold (III) chloride gel was investigated by performing inter-assay variation experiment and determining the relative standard deviation (R.S.D) or coefficient of variation between different batches of the gel. In this regard, 9 gel biosensors with a same configuration were made in total three batches (27 gold (III) chloride gel sensors) and treated with CTF spiked with different concentrations of AA (2000 μM, 500 μM and 50 μM) to account for variability. The inter-assay R.S.D. for n=3; samples=((2.949+8.506+5.939)/3)=5.798<10 reflects good reproducibility of results. The repeatability of the gold (III) chloride gel was investigated by performing an intra- and inter-day precision and accuracy analysis. The intra-day precision of the assay was estimated by calculating the relative standard (R.S.D) for the analysis of five replicates treated with CTF spiked with different concentrations of AA (2000 μM, 500 μM and 50 μM) and the inter-day precision was determined by analyzing three replicates treated with CTF spiked with different concentrations of AA (2000 μM, 500 μM and 50 μM) over three consecutive days. The accuracy was calculated based on the given formula ((mean determined concentration/nominal concentration)×100). The assay values on both the occasions (intra- and inter-day) were found to be within the accepted variable limits. The intra- and inter-day % accuracy values were in the range of 97.359-105.634 and 93.195-107.647 while the % precision values ranged from 3.488-7.288 and 5.039-8.674 respectively.

### Example 3

Validity of the Plasmonic Sensor and Methods

The validity of the results obtained from the gold (III) chloride gel is shown by the strong correlation with the AA concentration obtained through standard colorimetric coupled enzyme reaction assay and mass spectrometric based analytical methods. The gold (III) chloride gel provided accurate AA concentration within two minutes using 5 μL of sample, showing that laboratory quality data can be realized with this technique by using a simple gel.

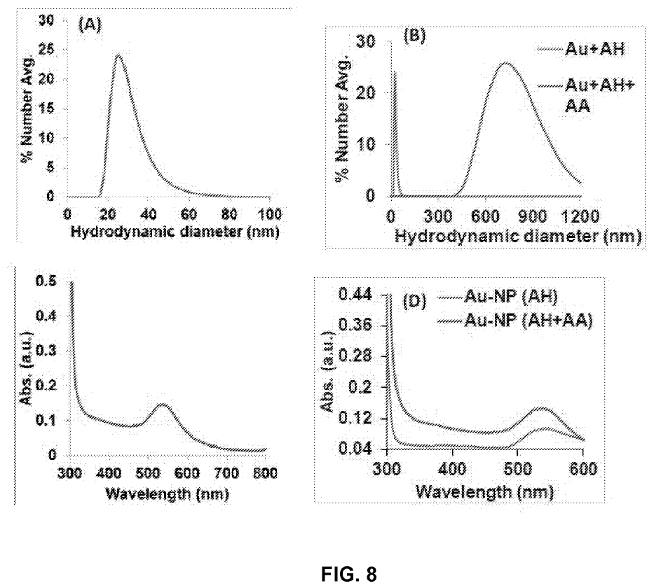

Initially, gold sol reducing ability of aqueous humor (AH) in solution phase was verified by generation of gold nanoparticles. It was followed by measurement of hydrodynamic diameter and surface Plasmon resonance. A 30±5 nm gold nanoparticle was produced by AH mediated reduction of gold sol in aqueous phase (FIG. 8A) and a significant change was reported when additional amount of AA was added (1 mM) to generate ˜700 nm assemblies (FIG. 8B), indicating clusters of aggregating gold nanoparticles. Study with UV-vis spectroscopy revealed the generation of characteristic Plasmon band at 560 nm post AA mediated reduction (FIG. 8C) which was retained with AH and enhanced by adding AA (FIG. 8D).

Visual Inspection of the Device Post Water, AH and AA Incubations

Visual inspections of the device post water, AH and AA incubations clearly showed the dark color circles at the site of sample doping in the device and intensity of circles were found directly proportional to concentration of AA in known samples and respective concentrations in AH samples (FIG. 8). Results could be visualized within two minutes of incubation started and did not change too much even in next 30 minutes.

### CONCLUSION

The methods and compositions of the disclosure can replace the subjective Seidel test that is currently the gold standard for evaluating aqueous humor leaks. This will offer many advantages to the ophthalmology community. The methods and compositions will be a game changer for the evaluation of post-surgical incisions from glaucoma filtering procedures (such as trabeculectomies) as well as for anterior ocular trauma patients.

It has been shown that ascorbic acid concentrations in the tear film are connected to release of the antioxidant from the lacrimal gland with tear film production and do not come from leaking of the molecule through the cornea in normal healthy eyes. Some AA may leak through the cornea, but not in higher concentrations than healthy tear film.

In a research sense, the biosensor offers a reliable, objective standard for grading the degree of a wound leak, which could be used to stratify wounds leaks into categories based on severity, with higher severity leaks seen in cases of higher AA concentration in the tear film. This will provide researchers with a reproducible way for monitoring post-operative wound leaks and could replace alternative methods that are currently used.

Finally, this reliable technology would also be able to revolutionize post-operative management in remote areas or third world countries where access to specialist is limited. In these situations the biosensor can be used by health care aids to monitor post-operative patients and used to help in the decision of whether treatment is needed. The biosensor will provide critical diagnostic information care providers in order to initiate sight-saving treatments.

In summary, eye conditions, including for example, a full-thickness laceration in the cornea or anterior scleral from trauma or incisional surgery releases aqueous humor into the tear film, which pathologically increases the concentration of tear film AA to a measurably higher level than that found in normal eyes. Ascorbic acid can also be released into the tear film from perforations in the cornea or anterior sderal from infections or ulcers that create full thickness perforations. This level can be detected with the use of the methods and compositions of the disclosure. The results from the biosensor can be used as a surrogate biomarker of the integrity of anterior ocular wounds and eye conditions. Current methodologies of absorption and fluorescence-based detection are not effective because they have low sensitivity and consume too much time to be clinically relevant in emergency or office settings. Biosensors of the disclosure can accurately and quantitatively measure AA levels in in vitro testing of aqueous humor samples collected from human subjects. The clinical samples from human aqueous humor were successfully tested to determine the concentration of AA. The methods and compositions of the disclosure provide a significant change in the current method for evaluating eye post-surgical patients as well as trauma patients. It will improve the utilization of health care resources and quality of care of patients.

We claim:

1. A biosensor for the detection of an analyte, the biosensor comprising:
a container or substrate; and
gold (III) chloride gel.

2. The biosensor of claim 1, wherein the analyte is ascorbic acid.

3. The biosensor of claim 1, wherein gold (III) chloride gel is formed from a gold chloride solution, and wherein the gold chloride solution has a concentration of between about 10 mM and about 50 mM.

4. The biosensor of claim 1, wherein the container or substrate is a tube, well, bottle, cylinder, dish, cup, bag, or channeled, textured, or fiat plastic or glass sheet.

5. A kit for detection of an analyte in a sample, the kit comprising:
the biosensor of claim 1; and
a collection container for collecting the sample.

6. The kit of claim 5, further comprising a color pixel code legend, wherein the color legend includes colors and respective analyte concentrations.

7. The kit of claim 5, further comprising a color pixel detection device.

8. The kit of claim 7, wherein the color detection device comprises a camera and a color picker tool.

9. A method of detecting an analyte in a sample, the method comprising:
contacting the biosensor of claim 1 with the sample;
determining an optical condition for the sample; and
detecting a concentration of the analyte in the sample based on the optical condition.

10. The method of claim 9, wherein the analyte is ascorbic acid.

11. The method of claim 9, wherein the optical condition is color intensity or color change.

12. The method of claim 9, wherein the sample is tears, tear film, aqueous humor, sweat, blood, serum, plasma, urine, saliva, or other bodily fluids.

13. The method of claim 9, wherein determining the optical condition of the sample occurs within between about one to about ten minutes of contacting the biosensor with the sample.

14. The method of any claim 9, wherein determining the optical condition of the sample comprises taking a picture of the gold chloride gel after being contacted with the sample.

15. The method of claim 9, wherein determining the optical condition of the sample comprises viewing the gold chloride gel after being contacted with the sample.

16. The method of claim 9, wherein the concentration of the analyte is reported on an electronic screen.

17. The method claim 9, wherein the concentration of the analyte in the sample indicates an eye condition, and wherein the eye condition is a full or partial thickness laceration or perforation to the anterior chamber of the eye, eye disease, mechanical or chemical eye injury, anterior scleral injury, corneal wound integrity, aqueous humor leaks, eye ulcer, infection, wound healing, or surgical incisions.

18. The method of claim 17, wherein the concentration of the analyte in the sample indicates severity of the eye condition or speed of wound healing.

19. A method of diagnosing an eye condition in a subject, the method comprising:
contacting the biosensor of claim 1 with a sample from the subject;
detecting a concentration of an analyte in the sample; and
diagnosing an eye condition in the subject where the concentration of analyte is elevated as compared to a control sample.

20. The method of claim 19, wherein the sample is tears, tear film, aqueous humor, sweat, blood, serum, plasma, urine, saliva, or other bodily fluids, and wherein the eye condition is a full or partial thickness laceration or perforation to the anterior chamber of the eye, eye disease, mechanical or chemical eye injury, anterior scleral injury, corneal wound integrity, aqueous humor leaks, eye ulcer, infection, wound healing, or surgical incisions.
